(19) United States
(12) Patent Application Publication
(10) Pub. No.: US 2019/0059838 A1
(43) Pub. Date: Feb. 28, 2019

(54) BREAST IMAGING APPARATUS AND APPARATUS AND METHOD FOR IMAGE PROCESSING

(71) Applicant: CANON KABUSHIKI KAISHA, Tokyo (JP)

(72) Inventor: Tetsuo Shimada, Hachioji-shi (JP)

(21) Appl. No.: 16/106,798

(22) Filed: Aug. 21, 2018

(30) Foreign Application Priority Data
Aug. 25, 2017 (JP) 2017-162616

(51) Int. Cl. A61B 6/00; A61B 6/04; G06T 7/00

(52) U.S. Cl. CPC A61B 6/502; G06T 2207/10116; G06T 7/0012; A61B 6/0414

(57) ABSTRACT

A breast imaging apparatus includes a gantry including a radiation generating unit and a radiation detecting unit configured to detect the radiation emitted from the radiation generating unit, wherein the radiation generating unit and the radiation detecting unit can be rotated in an opposed state, an extraction unit configured to extract first tissues and a second tissue from a three-dimensional image based on a projection image output from the radiation detecting unit, a classification unit configured to classify a group of the first tissues with reference to the second tissue, and a display configured to distinguishably display the group of the first tissues classified by the classification unit.

### BACKGROUND

### Field

The present disclosure relates to a breast imaging apparatus that extracts a specific tissue from an image acquired by capturing an image of a breast for display and an apparatus and a method for processing an image.

### Description of the Related Art

There are known breast imaging apparatuses that rotate a radiation generating unit for generating radiation and a radiation detecting unit for detecting the radiation with a rotating unit to capture an image of a breast. One example is a CT scanner configured such that the radiation generating unit and the radiation detecting unit, which are opposed with a rotation axis passing through an opening into which the breast is to be inserted therebetween, rotate about the rotation axis (for example, Japanese Patent Laid-Open No. 2010-068929). Another example is a technique for detecting a cluster of focuses (calcification) based on the three-dimensional position and associating individual focuses with the cluster of focuses (PCT Japanese Translation Patent Publication No. 2013-509229). Still another example is a technique for grouping pixel regions belonging to a corresponding region in a projection image in which a three-dimensional image is projected to one plane and displaying the group in a color or luminance according to the number of the pixel regions (for example, Japanese Patent Laid-Open No. 2016-22143).

The three-dimensional image formed using the above techniques can include a specific tissue (for example, microcalcified tissue). When a three-dimensional image including a specific tissue is projected for display, a plurality of tissues can overlap on the three-dimensional image according to the projection angle on the screen. This can make it difficult to grasp the size of the specific tissue and the relationship between the specific tissue and the other tissues.

### SUMMARY

Various embodiments of the present disclosure provide a breast imaging apparatus and an apparatus and a method for image processing capable of grasping the size of a specific tissue and the relationship between the specific tissue and other tissues in a projected three-dimensional image.

According to one embodiment of the present disclosure, a breast imaging apparatus includes a gantry including a radiation generating unit and a radiation detecting unit configured to detect the radiation emitted from the radiation generating unit, wherein the radiation generating unit and the radiation detecting unit can be rotated in an opposed state, an extraction unit configured to extract first tissues and a second tissue from a three-dimensional image based on a projection image output from the radiation detecting unit, a classification unit configured to classify a group of the first tissues with reference to the second tissue, and a display configured to distinguishably display the group of the first tissues classified by the classification unit.

Further features will become apparent from the following description of exemplary embodiments with reference to the attached drawings.

### BRIEF DESCRIPTION OF THE DRAWINGS

FIG. 1 is a diagram illustrating the appearance of a breast imaging apparatus according to an embodiment of the present disclosure.

FIG. 2 is a diagram illustrating the appearance of the breast imaging apparatus according to an embodiment of the present disclosure viewed from the CT imaging side.

FIG. 3 is a sectional view of the breast imaging apparatus according to an embodiment of the present disclosure.

FIG. 4 is a diagram illustrating the appearance of the breast imaging apparatus according to an embodiment of the present disclosure viewed from the mammography imaging side.

FIG. 5 is a diagram illustrating the configuration of the breast imaging apparatus according to an embodiment of the present disclosure.

FIG. 6 is a diagram illustrating the configuration of an image processing unit according to an embodiment of the present disclosure.

FIG. 7 is a diagram illustrating one classification form in which specific tissues extracted by an extraction section according to an embodiment of the present disclosure are classified.

FIG. 8 is a diagram illustrating one classification form in which specific tissues extracted by the extraction section according to an embodiment of the present disclosure are classified.

FIG. 9 is a diagram illustrating one classification form in which specific tissues extracted by the extraction section according to an embodiment of the present disclosure are classified.

FIG. 10 is a diagram illustrating one classification form in which specific tissues extracted by the extraction section according to an embodiment of the present disclosure are classified.

FIG. 11 is a diagram illustrating one classification form in which specific tissues extracted by the extraction section according to an embodiment of the present disclosure are classified.

FIG. 12 is a diagram illustrating one classification form in which specific tissues extracted by the extraction section according to an embodiment of the present disclosure are classified.

FIG. 13 is a diagram illustrating one display form of a display according to an embodiment of the present disclosure.

FIG. 14 is a diagram illustrating one display form of the display according to an embodiment of the present disclosure.

FIG. 15 is a diagram illustrating one display form of the display according to an embodiment of the present disclosure.

### DESCRIPTION OF THE EMBODIMENTS

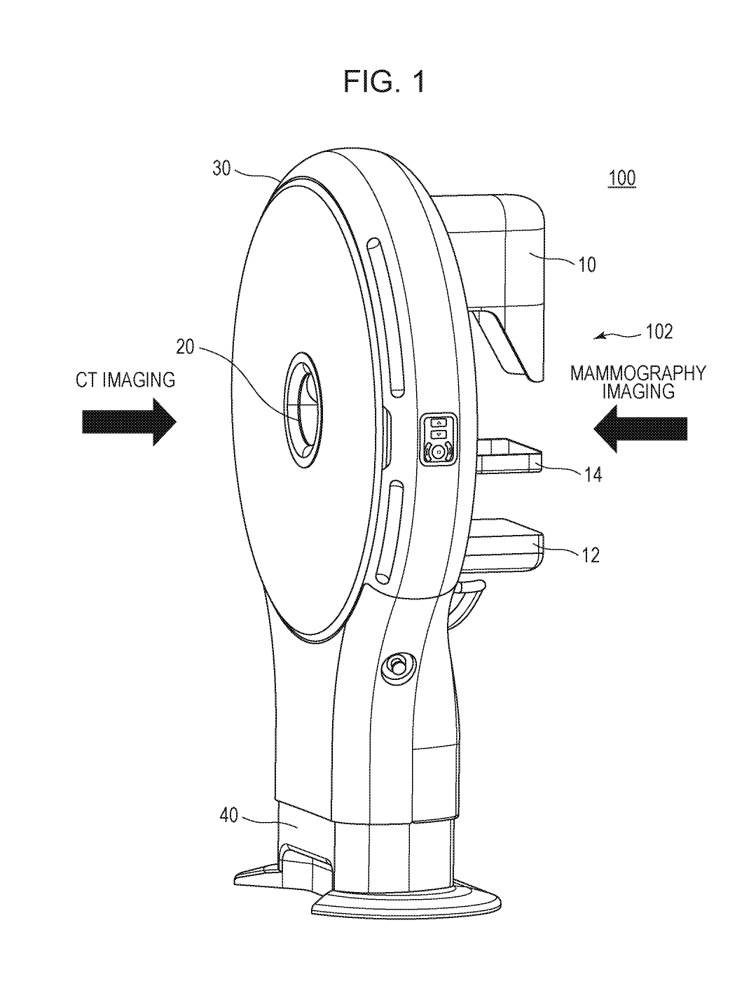

FIG. 1 is a diagram illustrating the appearance of a breast imaging apparatus 100. The breast imaging apparatus 100 is capable of mammography imaging and CT imaging, When a mammography image and a CT image are to be captured, the subject is in the standing position. In the standing position, the subject is standing on the floor with both feet on the floor.

The breast imaging apparatus 100 is configured so as to rotate a radiation generating unit 10 that generates radiation and a radiation detecting unit 12 for detecting the radiation emitted from the radiation generating unit 10 in an opposed state. An image capturing unit 102 mainly includes the radiation generating unit 10 and the radiation detecting unit 12.

The image capturing unit 102 captures an image of the image-capturing target part (breast) of the subject from a first side of the breast imaging apparatus 100, with the target part sandwiched between a pressing plate 14 and the radiation detecting unit 12. In other words, the breast imaging apparatus 100 has a mammography imaging mode. The image-capturing target part (breast) of the subject is inserted between the radiation generating unit 10 and the radiation detecting unit 12 onto a breast cup from a second side of the breast imaging apparatus 100 opposite to the first side, and in that state, an image of the target part is captured by rotating the radiation generating unit 10 and the radiation detecting unit 12. In other words, the breast imaging apparatus 100 has a CT imaging mode.

For the mammography imaging, the image-capturing target part (breast) of the subject is photographed from the first side (the right in the drawing) of the breast imaging apparatus 100, with the target part sandwiched between the pressing plate 14 and the radiation detecting unit 12. The pressing plate 14 is made of a transparent material that transmits radiation. Specifically, the breast of the subject can be sandwiched between the pressing plate 14 and the radiation detecting unit 12 by vertically moving the pressing plate 14. With the subject's breast sandwiched between the pressing plate 14 and the radiation detecting unit 12, the radiation generating unit 10 emits radiation. An image of the subject's breast can be captured by detecting the radiation that has passed through the subject's breast with the radiation detecting unit 12. The breast imaging apparatus 100 can create a mammographic image based on the acquired radiation data.

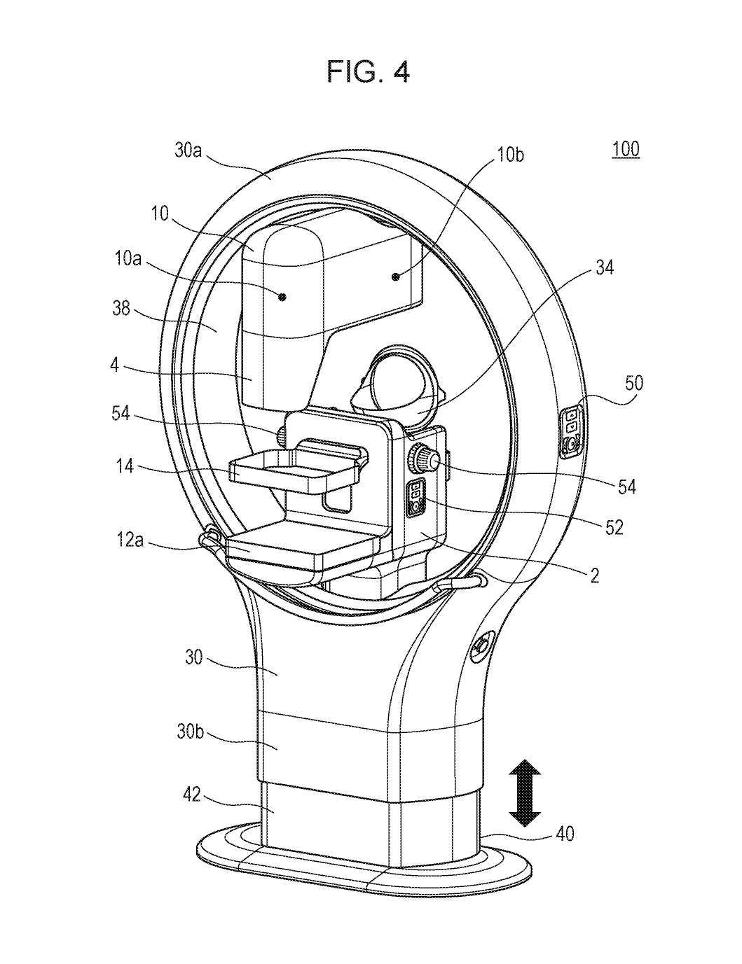

For CT imaging, the image-capturing target part (breast) of the subject is inserted between the radiation generating unit 10 and the radiation detecting unit 12 from the second side (the left in the drawing) opposite to the first side of the breast imaging apparatus 100. In that state, the target part is photographed by rotating the radiation generating unit 10 and the radiation detecting unit 12 with a rotating frame 38 (see FIG. 4). Specifically, a gantry 30 of the breast imaging apparatus 100 has an opening 20 into which the subject's breast is to be inserted. The radiation generating unit 10 and the radiation detecting unit 12 are rotated with the rotating frame 38, with the subject's breast inserted in the opening 20 and placed on the breast cup, to capture an image of the breast. The radiation generating unit 10 generates radiation while the radiation generating unit 10 and the radiation detecting unit 12 are being rotated with the rotating frame 38. The radiation detecting unit 12 can capture an image of the subject's breast by detecting the radiation that has passed through the subject's breast. The breast imaging apparatus 100 can create a CT image by reconstructing the acquired radiation data.

The first side of the breast imaging apparatus 100 is a mammography imaging side, and the second side of the breast imaging apparatus 100 is a CT imaging side. The line connecting the first side (mammography imaging side) and the second side (CT imaging side) horizontally is substantially parallel to the rotation shaft of the rotating frame 38. The line connecting the first side (mammography imaging side) and the second side (CT imaging side) horizontally is orthogonal to the planar surface of the substantially planer gantry 30, or the planar surface of the front cover 26.

The first side (mammography imaging side) and the second side (CT imaging side) of the breast imaging apparatus 100 are divided by the substantially planar gantry 30, the front cover 26, and the image capturing unit 102 of the breast imaging apparatus 100.

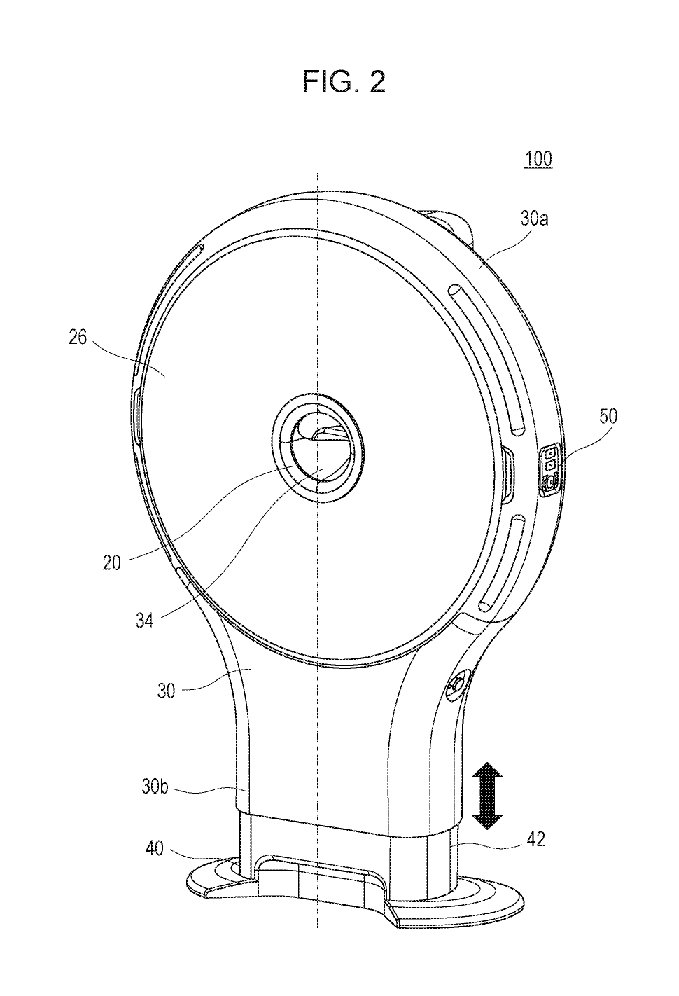

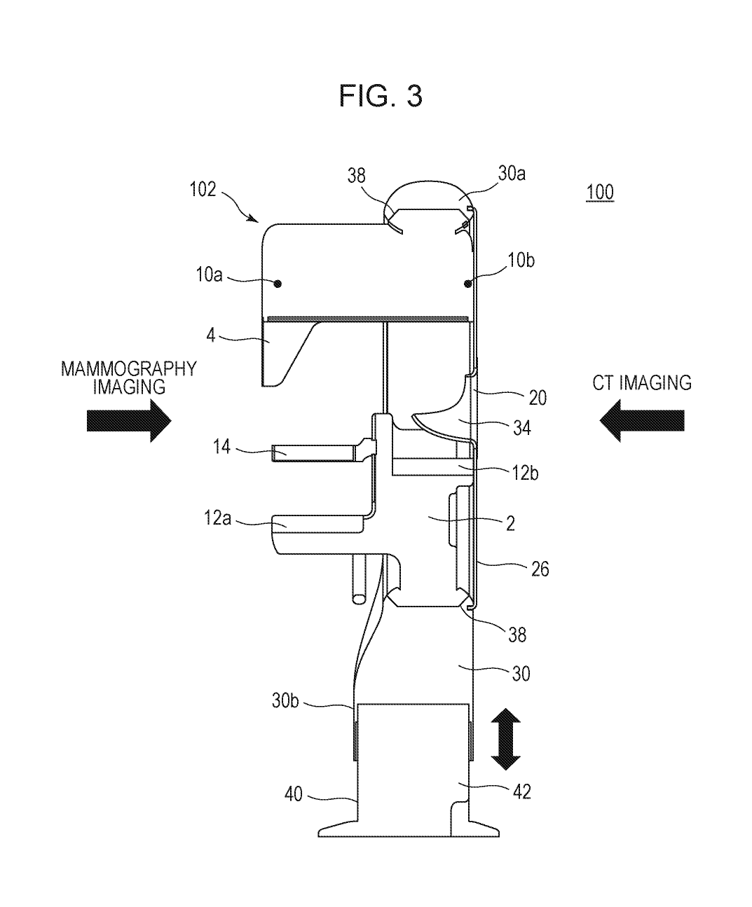

Referring to FIGS. 2 to 4, the breast imaging apparatus 100 will be specifically described. FIG. 2 is a diagram illustrating the appearance of the breast imaging apparatus 100 viewed from the CT imaging side. FIG. 3 is a sectional view of the breast imaging apparatus 100. The sectional view of the breast imaging apparatus 100 is taken along the center line (a chain line) of the breast imaging apparatus 100 extending in the vertical direction in FIG. 2. FIG. 4 is a diagram illustrating the appearance of the breast imaging apparatus 100 viewed from the mammography imaging side.

As illustrated in FIG. 2, the gantry 30 includes, at the CT imaging side, a front cover 26 for protecting the subject from the radiation generating unit 10 and the radiation detecting unit 12 which rotate during CT scanning. The front cover 26 has the opening 20 into which the breast of the CT subject is to be inserted. A breast cup 34 is continuously provided from the opening 20 seamlessly.

As illustrated in FIG. 4, a pressing plate 14 for pressing the breast of the mammography subject is disposed at the mammography imaging side of the gantry 30, Furthermore, a protection plate 4 for protecting the subject from unnecessary exposure is disposed on the mammography imaging side of the gantry 30.

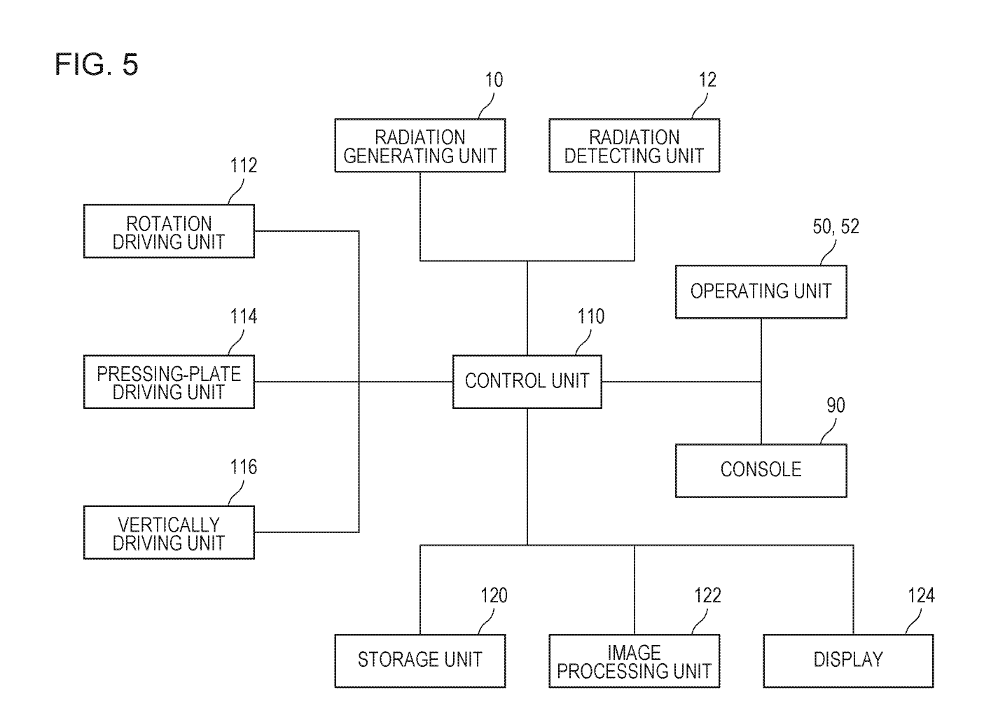

FIG. 5 illustrates the configuration of the breast imaging apparatus 100. The breast imaging apparatus 100 includes a rotation driving unit 112 that rotates the radiation generating unit 10 and the radiation detecting unit 12 in an opposed state. The breast imaging apparatus 100 further includes a pressing-plate driving unit 114 that moves the pressing plate 14 vertically and a vertically driving unit 116 that moves the gantry 30 vertically with respect to a support leg 40.

The breast imaging apparatus 100 further includes a control unit 110 that controls the radiation generating unit 10, the radiation detecting unit 12, the rotation driving unit 112, the pressing-plate driving unit 114, and the vertically driving unit 116. The breast imaging apparatus 100 further includes operating units 50 and 52 and a console 90 that transmit instructions to the control unit 110. An operating unit 50 that operates the breast imaging apparatus 100 is disposed on the gantry 30. The operating unit 52 having the same function as the function of the operating unit 50 is disposed on a support base 2 that supports the radiation detecting unit 12. The console 90 is disposed outside the imaging room.

The breast imaging apparatus 100 further includes a storage unit 120 that stores images, such as projection images, tomographic images, and three-dimensional images formed of a plurality of tomographic images, an image processing unit 122 that creates a tomographic image, which is a two-dimensional image, and a three-dimensional image from volume data obtained by reconstructing a plurality of projection images, and a display 124 that displays the tomographic image and the three-dimensional image. The projection images obtained from the radiation detecting unit 12 are stored in the storage unit 120. The image processing unit 122 reads the plurality of projection images and reconstructs the images into volume data. The storage unit 120 stores the volume data reconstructed by the image processing unit 122. The display 124 reads the volume data from the storage unit 120 according to an instruction from the operator and displays a tomographic image. The display 124 can also display a three-dimensional image.

The radiation generating unit 10 mainly includes an electron emission source that emits electrons and a target (not illustrated). The electrons emitted from the electron emission source are radiated toward the target due to the potential difference between the cathode and the anode. The target is a member that generates radiation by collision of electrons. The radiation emitted from the target is radiated to the outside in a cone beam shape. The control unit 110 can control the image capturing conditions of the radiation generating unit 10.

The radiation detecting unit 12 detects the radiation that has passed through the subject with a photoelectric conversion element and outputs the radiation as an electrical signal. For example, the radiation detecting unit 12 includes a conversion panel that detects the radiation that has passed through the subject, a capacitor, and an interface (LT) for outputting information obtained by converting the radiation to an electrical signal. The electrical signal is output to the control unit 110 via the FF.

As illustrated in FIGS. 2 to 4, the gantry 30 includes the ring-shaped rotating frame 38 for rotating the radiation generating unit 10 and the radiation detecting unit 12 in an opposed state and a ring-shaped fixing frame 30a that rotatably supports the rotating frame 38. The gantry 30 further includes a long cylindrical portion 30b connected to the fixing frame 30a. The rotating frame 38 and the fixing frame 30a can also be referred to as a rotating unit that rotates the radiation generating unit 10 and the radiation detecting unit 12. The fixing frame 30a and the long cylindrical portion 30b are unitary formed. The fixing frame 30a is positioned above the long cylindrical portion 30b. The long cylindrical portion 30b is connected to the support leg 40 that supports the gantry 30 with respect to the floor surface.

The gantry 30 is vertically erected to allow image-capturing, with the subject in the standing position. The rotation axis of the rotating unit (the rotating frame 38 of the gantry 30) that rotates the radiation generating unit 10 and the radiation detecting unit 12 is in the horizontal direction.

The long cylindrical portion 30b covers the outer circumference of a long cylindrical portion 42 of the support leg 40. In other words, the long cylindrical portion 42 of the support leg 40 is fitted in the long cylindrical portion 30b of the gantry 30, so that the long cylindrical portion 42 of the support leg 40 and the long cylindrical portion 30b of the gantry 30 are nested.

The breast imaging apparatus 100 includes the vertically driving unit 116 that moves the long cylindrical portion 30b vertically with respect to the support leg 40 to allow adjusting the height of the opening 20 according to the body shape of the subject.

The breast imaging apparatus 100 includes the radiation generating unit 10 that generates radiation and the radiation detecting unit 12 for detecting the radiation emitted from the radiation generating unit 10. The radiation generating unit 10 and the radiation detecting unit 12 can be rotated in an opposed state.

The radiation generating unit 10 and the radiation detecting unit 12 are disposed on the rotating frame 38 that rotates relative to the fixing frame 30a of the gantry 30. In this case, as illustrated in FIG. 3, the breast imaging apparatus 100 includes a radiation generating section 10b and a radiation detecting section 12b for CT imaging and a radiation generating section 10a and a radiation detecting section 12a for mammography imaging. The gantry 30 includes the radiation generating section 10b and the radiation detecting section 12b for CT imaging and the radiation generating section 10a and the radiation detecting section 12a for mammography imaging. In other words, the breast imaging apparatus 100 includes two sets of radiation generating sections and radiation detecting sections for CT imaging and mammography imaging.

The gantry 30 includes the ring-shaped rotating frame 38 for rotating the radiation generating section 10b and the radiation detecting section 12b for CT imaging and the radiation generating section 10a and the radiation detecting section 12a for mammography imaging in the opposed state.

Specifically, for CT imaging, the radiation generating section 10b and the radiation detecting section 12b are disposed at the rotating frame 38. The radiation detecting section 12b is disposed at the rotating frame 38 via the support base 2 that supports the radiation detecting section 12b.

For mammography imaging, the radiation generating section 10a and the radiation detecting section 12a are disposed at the rotating frame 38. The radiation detecting section 12a is disposed at the rotating frame 38 via the support base 2.

The rotating frame 38 is connected to the fixing frame 30a of the gantry 30 via a bearing with a bearing structure. The fixing frame 30a is in a stationary state and is a stationary frame. The rotating frame 38 can be rotated by the rotation driving unit 112. The rotation driving unit 112 is disposed in the gantry 30 so that the rotation axis of the rotating frame 38 is in the horizontal direction.

The pressing plate 14 is disposed on the support base 2 so as to be vertically movable. Rotating tubs 54 for vertically moving the pressing plate 14 are disposed on the support base 2. By rotating the rotating tub 54 to move the pressing plate 14 downward, the subject's breast can be sandwiched between the pressing plate 14 and the radiation detecting section 12a.

Thus, the support base 2 is disposed on the rotating frame 38 and supports the radiation detecting section 12a, the radiation detecting section 12b, and the pressing plate 14. By rotating the rotating frame 38 together with the support base 2 with the rotation driving unit 112, the radiation detecting section 12a and the radiation detecting section 12b can be rotated. By rotating the rotating frame 38 with the rotation driving unit 112, the radiation generating section 10a and the radiation generating section 10b can be rotated.

As illustrated in FIG. 3, the radiation generating section 10a and the radiation generating section 10b are disposed at substantially the same height. The radiation detecting section 12b is disposed at a position higher than the radiation detecting section 12a.

In other words, the radiation generating section 10a and the radiation generating section 10b are disposed at the same position (the same distance) with respect to the rotation axis of the rotating unit (the rotating frame 38).

The radiation detecting section 12a and the radiation detecting section 12b are disposed such that the radiation detecting section 12a is positioned outer than the radiation detecting section 12b with respect to the rotation axis of the rotating unit (the rotating frame 38).

The distance between the radiation generating section 10b and the radiation detecting section 12b for use in CT imaging is shorter than the distance between the radiation generating section 10a and the radiation detecting section 12a for use in mammography imaging.

For mammography imaging, the subject's breast is sandwiched between the pressing plate 14 and the radiation detecting section 12a. Since the subject's breast is pressed by the pressure into a flat shape, the radiation irradiation area has to be increased to ensure a sufficient field of view (FOV). For that purpose, the radiation detecting section 12a for use in mammography imaging is disposed at a position lower the radiation detecting section 12b for use in CT imaging.

The field of view 8 is a field of view offered by the radiation generating section 10a for mammography imaging. The radiation generating section 10a and the radiation detecting section 12a are disposed so that the field of view 8 of the radiation generating section 10a includes the pressing plate 14. The field of view 8 has a quadrangular pyramid shape (a cone beam shape) expanding with the focus of the radiation generating section 10b as the apex. As illustrated in FIG. 3, one end (the left side) of the field of view 8 is vertical, and the other end (the right side) of the field of view 8 is oblique. To capture an image of the periphery of the subject's breast (axilla), the field of view 8 of the radiation generating section 10a is set so that an end of the field of view 8 adjacent to the subject of the mammography (the left side) (an end or an end face of the field of view) is vertical.

In contrast, for CT imaging, the radiation generating section 10b and the radiation detecting section 12b are disposed so that the rotating frame 38 and the entire breast imaging apparatus 100 (gantry 30) are compact. Specifically, the radiation generating section 10h and the radiation detecting section 12b are disposed so that the distance between the radiation generating section 10b and the radiation detecting section 12b is as short as possible. The radiation detecting section 12b is disposed directly under the breast holding portion 34. The radiation detecting section 12b is disposed at a position where the radiation detecting section 12b is not brought into contact with the breast holding portion 34 even when the radiation detecting section 12b is rotated by the rotating frame 38.

The field of view 6 is a field of view offered by the radiation generating section 10a for CT imaging. The subject's breast undergoing CT imaging is held on the breast holding portion 34 and is not pressed. The radiation generating section 10b and the radiation detecting section 12b are disposed so that the field of view 6 of the radiation generating section 10b includes the end of the breast holding portion 34.

The field of view 6 has a quadrangular pyramid shape (a cone beam shape) expanding with the focus of the radiation generating section 10b as the apex. As illustrated in FIG. 3, one end (the right side) of the field of view 6 is vertical, and the other end (the left side) of the field of view 6 is oblique. To capture an image of the periphery of the subject's breast (axilla), the field of view 6 of the radiation generating section 10b is set so that an end of the field of view 6 adjacent to the subject of the mammography (the left side) (an end or an end face of the field of view) is vertical.

Thus, the field of view 8 is set so that an end of the field of view 8 adjacent to the subject of the mammography (the left side) (an end or an end face of the field of view) is vertical, and the field of view 6 is set so that an end of the field of view 6 adjacent to the subject of the CT imaging (the right side) (an end or an end face of the field of view) is vertical. Breast cancer can metastasize to the periphery of the breast (axilla). The field of view 6 of the radiation generating section 10b for CT imaging and the field of view 8 of the radiation generating section 10a for mammography imaging are individually set so that an image of the periphery of the subject's breast (axilla) can be captured.

For CT imaging, the radiation generating section 10b for CT imaging may be disposed higher than the radiation generating section 10a for mammography imaging to ensure a sufficient field of view. In CT imaging, the radiation generating section 1011 and the radiation detecting section 12b rotate while the radiation generating section 10b is emitting radiation from the focus.

Thus, the breast imaging apparatus 100 according to an embodiment of the present disclosure includes the first radiation generating section 10a that generates radiation and the second radiation generating section 10b that generates radiation. The breast imaging apparatus 100 further includes the first radiation detecting section 12a for detecting the radiation emitted from the first radiation generating section 10a and the second radiation detecting section 12b for detecting the radiation emitted from the second radiation generating section 10b.

The breast imaging apparatus 100 captures an image of the image-capturing target part of the subject from the first side of the breast imaging apparatus 100, with the target part sandwiched between the pressing plate 14 and the first radiation detecting section 12a, using the first radiation generating section 10a and the first radiation detecting section 12a. The breast imaging apparatus 100 captures an image of the image-capturing target part of the subject from the second side opposite to the first side, with the target part inserted between the second radiation generating section 10b and the second radiation detecting section 12b, while rotating the second radiation generating section 10b and the second radiation detecting section 12b.

Thus, the breast imaging apparatus 100 includes the two radiation generating units for CT imaging and mammography imaging. This ensures a field of view suitable for each of the subject's breast undergoing CT imaging and the subject's breast undergoing mammography imaging,

The breast imaging apparatus 100 includes the rotation driving unit 112 that rotates the radiation generating unit 10 and the radiation detecting unit 12 via the rotating frame 38. The radiation generating unit 10 includes therein the radiation generating section 10a for mammography imaging and the radiation generating section 10b for CT imaging.

FIG. 4 illustrates a form for mammography imaging in a craniocaudal view (CC) with the breast imaging apparatus 100. The position of the rotating frame 38 is set so that the radiation generating section 10a, the pressing plate 14, and the radiation detecting section 12a are arranged in the vertical direction.

The distance between the pressing plate 14 and the radiation detecting section 12a can be adjusted by rotating the rotating tubs 54 to move the pressing plate 14. The movement of the pressing plate 14 allows pressing the subject's breast. In the mammography imaging in the CC illustrated in FIG. 4, the breast disposed between the pressing plate 14 and the radiation detecting section 12a is compressed between the pressing plate 14 and the radiation detecting section 12a and is radiographed.

The rotation driving unit 112 is disposed inside the fixing frame 30a. The rotating frame 38 is rotatably connected to the rotation driving unit 112 via a connecting member (for example, a belt). A bearing is disposed in the gap between the fixing frame 30a and the rotating frame 38. The rotating frame 38 rotates with respect to the fixing frame 30a by the driving of the rotation driving unit 112.

For CT imaging, the breast imaging apparatus 100 can capture CT images of the subject's breast by detecting radiation that has passed through the subject's breast with the radiation detecting unit 12.

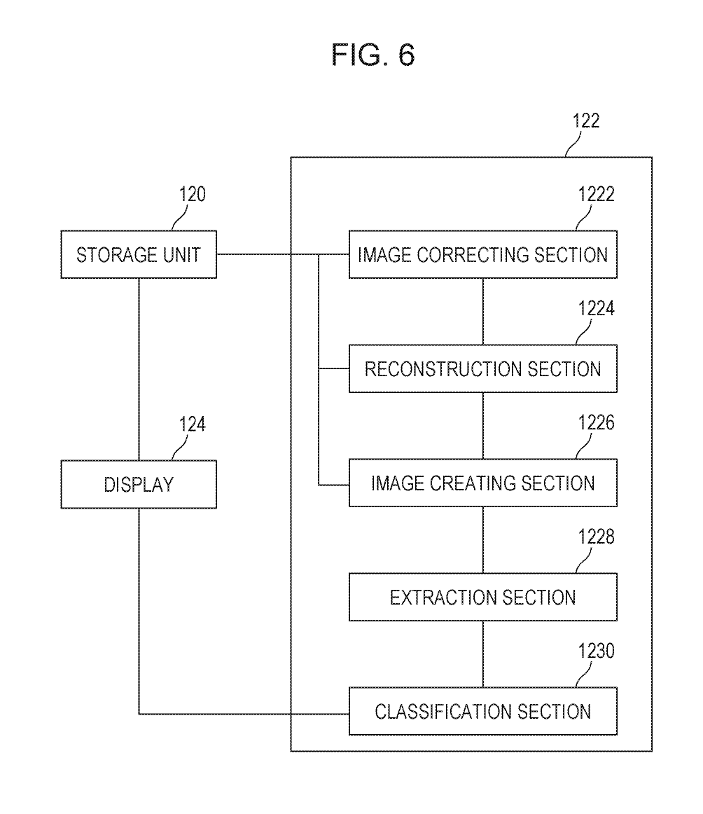

Referring to FIG. 6, the image processing unit 122 will be described. FIG. 6 is a diagram illustrating the configuration of the image processing unit 122. The image processing unit 122 includes an image correcting section 1222 that corrects projection images, a reconstruction section 1224 that reconstructs a plurality of projection images to generate volume data, and an image creating section 1226 that creates a three-dimensional image formed of a plurality of tomographic images based on the volume data. The image processing unit 122 further includes an extraction section 1228 that extracts first tissues and a second tissue from the three-dimensional image based on the projection images output from the radiation detecting section 12b and a classification section 1230 that classifies a group of first tissues with reference to the second tissue extracted by the extraction section 1228. The classification section 1230 outputs a first tissue group distinguishably. In other words, the classification section 1230 can output information indicating the size of the first tissue group relative to the second tissue.

The image correcting section 1222 obtains a gain correction image based on the signal of each pixel of the radiation detecting section 12b that detects radiation emitted from the radiation generating section 10b in a state no subject is present. Furthermore, the image correcting section 1222 obtains an offset correction image based on the signal of each pixel of the radiation detecting section 12b without radiation emitted from the radiation generating section 10b. In the case where the image correcting section 1222 cannot capture one or both of the gain correction image and the offset correction image before capturing of an image of the subject, the image correcting section 1222 may capture the one or both of the images after image capturing. The image correcting section 1222 corrects the projection images of the subject using the gain correction image and the offset correction image to create corrected projection images.

The projection images corrected by the image correcting section 1222 are stored in the storage unit 120. The projection images stored in the storage unit 120 are transferred to the reconstruction section 1224 of the image processing unit 122. The projection images captured by the radiation detecting section 12b may be directly transferred to the reconstruction section 1224.

The reconstruction section 1224 reconstructs the plurality of projection images of the periphery of the subject's breast output from the radiation detecting section 12b to obtain volume data. The plurality of projection images may be projection images corrected by the image correcting section 1222. Known examples of a method of reconstruction include a filtered back projection method, a Feldkamp method in which the filtered back projection method is applied to multislices, and a successive approximation method. However, the present disclosure does not depend on these reconstruction methods. The volume data reconstructed by the reconstruction section 1224 is stored in the storage unit 120.

The image creating section 1226 creates a tomographic image from the volume data reconstructed by the reconstruction section 1224. Specifically, the image creating section 1226 creates a tomographic image by cutting out one cross section from the reconstructed volume data. The image creating section 1226 may create a plurality of tomographic images of orthogonal cross section. The image creating section 1226 can also create a tomographic image of axial section, sagittal section, or coronal section. The image creating section 1226 can also create a tomographic image of a desired cross section by setting the coordinates of the cross section with the console 90. The operator can create a tomographic image containing the region of interest (for example, a mammary gland) of the subject's breast by setting the coordinates of the cross section with the console 90 so that the region of interest is displayed.

The image creating section 1226 further creates a three-dimensional image by rendering the volume data. The rendering is a process of expressing the volume data in two dimensions in order to display the volume date on the screen of the display 124. In the rendering, the three-dimensional coordinates of the volume data are converted to two-dimensional coordinates, and a shadowing process for giving a stereoscopic effect is performed. Examples of the rendering include volume rendering and surface rendering. The image creating section 1226 may create a three-dimensional image as a raysum (ray-summation) image. The image creating section 1226 may create a three-dimensional image from a plurality of tomographic images.

The extraction section 1228 extracts specific tissues from the three-dimensional image output from the image creating section 1226. Examples of the specific tissues include calcified tissues and tumor tissues of the subject's breast. Here, the first tissue is assumed to be a calcified tissue, and the second tissue is assumed to be s tumor tissue. The extraction section 1228 may extract the calcified tissue and the tumor tissue for each tomographic image or from a rearranged tomographic image, such as an orthogonal cross section.

In this case, the extraction section 1228 extracts calcified tissue from a three-dimensional image using the characteristics that the CT values around the calcified tissue concentrically spread. The extraction section 1228 extracts calcified tissues from a three-dimensional image using a method for extracting calcified tissues using a fixed threshold, a method of extraction using a local threshold for peripheral pixels, and a method using a ring-shaped filter using a concentration gradient in which the CT values increase from the periphery to the center of the calcified tissue.

The extraction section 1228 analyzes the CT values in the three-dimensional image to specify a position at which the CT value is relatively high. The extraction section 1228 calculates the gradient of the CT values in three dimensions with the position at which the CT value is relatively high as the center. The gradient of the CT values is used to ascertain the situation in which the CT values around a calcified tissue concentrically spread. The extraction section 1228 extracts a calcified tissue from the information on the position where the CT value is relatively high and the gradient of the CT values. If the CT values around the position where the CT value is relatively high have a gentle three-dimensional gradient, the tissue is extracted as a calcified tissue. If the CT values around the position where the CT value is relatively high have not a gentle three-dimensional gradient, the tissue is not extracted as a calcified tissue.

In the vicinity of the center of calcified tissue, the CT value in the three-dimensional image takes a local maximum value. The extraction section 1228 can also extract calcified tissue using a position where the CT value takes a local maximum value. The extraction section 1228 calculates the gradient of the CT values with a position where the CT value takes the local maximum value as the center. The extraction section 1228 extracts calcified tissue from the information on the position where the CT value takes the local maximum value and the gradient of the CT values. The extraction section 1228 may also calculate the gradient of the CT values in the three-dimensional image in three dimensions as a vector and extract a calcified tissue from the status of the calculated vector.

In contrast, tumor tissue is a tissue whose CT value is lower than the CT values of the surroundings and higher than the CT value of calcified tissue. The extraction section 1228 analyzes the CT values in the three-dimensional image to extract tumor tissue using the characteristics that tumor tissue has a predetermined size (for example, a radius of a or more). The extraction section 1228 extracts a region having a CT value less than the values of the surroundings and having a predetermined size (area) as a tumor tissue.

Here, the extraction section 1228 may extract a tumor tissue using a region growing process for extracting a region having a predetermined size, a pattern matching process for extracting the region by referring to the characteristics of tumor tissue obtained in the past, or a differential filtering process for extracting tumor tissue by determining the difference between an image captured by CT imaging and an image having no tumor tissue.

Thus, the extraction section 1228 uses different methods in extracting local calcified tissues (the first tissues) and a tumor tissue (the second tissue) having a predetermines size. The extraction section 1228 may extract specific tissues using not the CT values but pixel values,

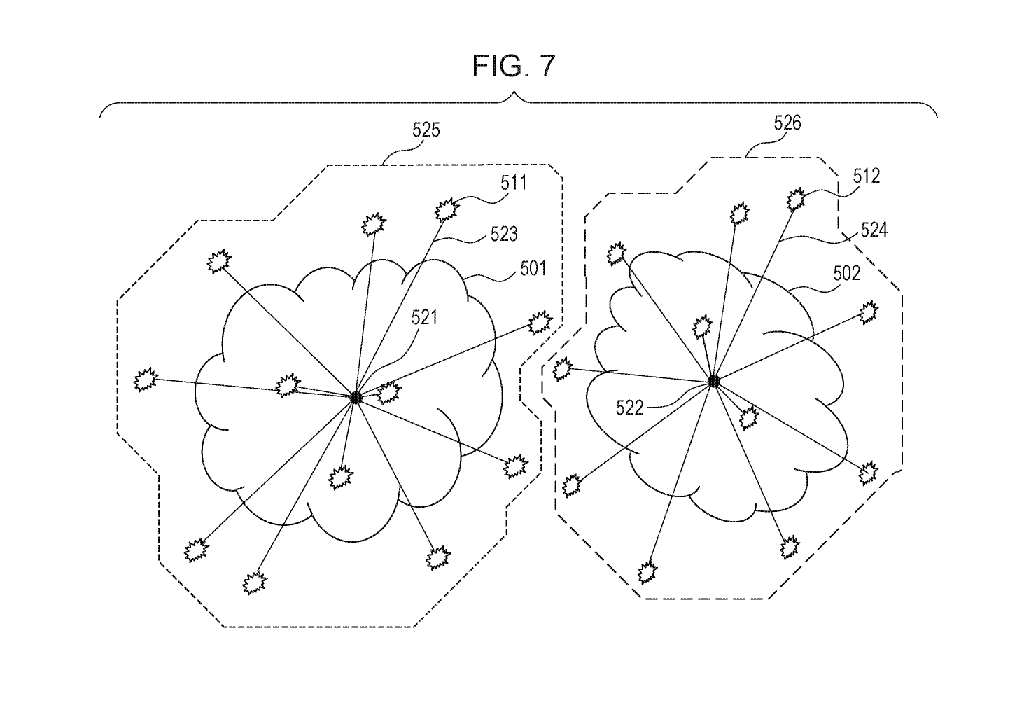

The classification section 1230 classifies the specific tissues extracted by the extraction section 1228. FIGS. 7 to 12 illustrate forms in which the specific tissues extracted by the extraction section 1228 are classified.

FIG. 7 illustrates one classification form in which the calcified tissues (the first tissues) and the tumor tissues (the second tissues) extracted by the extraction section 1228 are classified.

As illustrated in FIG. 7, a tumor tissue 501, a calcified tissue 511, a tumor tissue 502, and a calcified tissue 512 are extracted by the extraction section 1228. The classification section 1230 classifies the plurality of calcified tissues into a plurality of groups by the position of the plurality of calcified tissues. In this case, there are a group of calcified tissues including the calcified tissue 511 illustrated at the left in FIG. 7 and a group of calcified tissues including the calcified tissue 512 illustrated at the right in FIG. 7. There are a plurality of calcified tissues in addition to the calcified tissue 511 and the calcified tissue 512.

A method for classifying a plurality of calcified tissues to a plurality of groups includes a method of classification according to the distance from a specified position. As illustrated in FIG. 7, a specified position 521 and a specified position 522 are specified. The operator specifies the specified position 521 corresponding to the tumor tissue 501 and the specified position 522 corresponding to the tumor tissue 502 in the three-dimensional image via the console 90 (a specification unit) while viewing a three-dimensional image in which the tumor tissue 501 and the tumor tissue 502 are displayed. Thus, the operator can specify the positions of the tumor tissue 501 (the second tissue) and the tumor tissue 502 (the second tissue) in the three-dimensional image while viewing the tumor tissue 501 and the tumor tissue 502 in the three-dimensional image.

Alternatively, the classification section 1230 may specify the specified position 521 and the specified position 522 from the structures (forms) of the tumor tissue 501 and the tumor tissue 502 extracted by the extraction section 1228. Specifically, the classification section 1230 specifies the central point or the center of gravity of the tumor tissue 501 as the specified position 521, and the central point or the center of gravity of the tumor tissue 502 as the specified position 522. Thus, the specified position 521 and the specified position 522 may be specified using the method (program) of image processing for extracting the structures (forms) of the tumor tissue 501 and the tumor tissue 502.

The classification section 1230 searches for the calcified tissue 511 (the first tissue) based on the distance from the specified position 521. The classification section 1230 determines whether the distance between the specified position 521 and the calcified tissue 511 is within a predetermined range. The classification section 1230 determines whether the distance between the specified position 521 and a calcified tissue other than the calcified tissue 511 is within a predetermined range. If the classification section 1230 determines that the distance between the specified position 521 and the calcified tissue is within a predetermined range, then the classification section 1230 recognizes that the calcified tissue is of the same group and classifies the calcified tissue as a group 525.

Likewise, the classification section 1230 searches for the calcified tissue 512 (the first tissue) based on the distance from the specified position 522. The classification section 1230 determines whether the distance between the specified position 522 and the calcified tissue 512 is within a predetermined range. The classification section 1230 determines whether the distance between the specified position 522 and a calcified tissue other than the calcified tissue 512 is within a predetermined range. If the classification section 1230 determines that the distance between the specified position 522 and the calcified tissue is within a predetermined range, then the classification section 1230 recognizes that the calcified tissue is of the same group and classifies the calcified tissue as a group 526.

The distances for classifying the group of the calcified tissue 511 and the group of the calcified tissue 512 can be set to any distance via the console 90 (the specification unit). When the same calcified tissue is classified as the group 525 and the group 526 by the classification section 1230, the calcified tissue is classified to a group in which the distance between the specified position and the calcified tissue is smaller than that of the other group.

Euclidean distance, Mahalanobis distance, or the like may be used in obtaining the distance between the specified position and the calcified tissue. The Mahalanobis distance is a distance scaled from, for example, information on the long axis and the short axis of the tumor tissue, information on the running of the mammary gland, or the shape of the entire breast. The number of calcified tissues classified into a plurality of groups may be set in advance. For example, in the case where the number of calcified tissues is set to 10, the classification section 1230 searches for calcified tissues in order of ascending distance from the specified position 521 until ten calcified tissues are collected. In the case where the number of calcified tissues is set to 20, the classification section 1230 searches for calcified tissues in order of ascending distance from the specified position 521 until 20 calcified tissues are collected. The classification section 1230 may classify the groups using a cluster analysis in which specified positions and the number are not defined in advance.

Thus, the classification section 1230 classifies the group of calcified tissues (the first tissue) based on the preset distance or number. The classification section 1230 classifies the group of the calcified tissue 511 (the first tissue) as the group 525 (a first group) with reference to the tumor tissue 501 (the second tissue). The classification section 1230 classifies the group of the calcified tissue 512 (the first tissue) as the group 526 (a second group) with reference to the tumor tissue 502 (the second tissue).

The classification section 1230 outputs information indicating the group 525 of the calcified tissue 511 in the tumor tissue 501 and information indicating the group 526 of the calcified tissue 512 in the tumor tissue 502 to the display 124. The display 124 distinguishably displays the group 525 of the calcified tissue 511 in the tumor tissue 501 and the group 526 of the calcified tissue 512 in the tumor tissue 502. In other words, the display 124 distinguishably displays the group 525 (the first group) of the calcified tissue 511 and the group 526 (the second group) of the calcified tissue 512, which are respectively extracted with reference to the tumor tissue 501 and the tumor tissues 502 (the plurality of second tissues).

For example, the display 124 displays the group 525 of the calcified tissue 511 in the tumor tissue 501 in blue and displays the group 526 of the calcified tissue 512 in the tumor tissue 502 in red. Alternatively, the display 124 displays the group 525 of the calcified tissue 511 in the tumor tissue 501 with a thick frame (a solid line) and displays the group 526 of the calcified tissue 512 in the tumor tissue 502 with a thin frame (a broken line).

Since the display 124 distinguishably displays the group 525 of the calcified tissue 511 in the tumor tissue 501 and the group 526 of the calcified tissue 512 in the tumor tissue 502, the operator can ascertain the size of the calcified tissue in each tumor tissue. In other words, the operator can ascertain the size of the specific tissue and the relationship between the specific tissue and the other tissue.

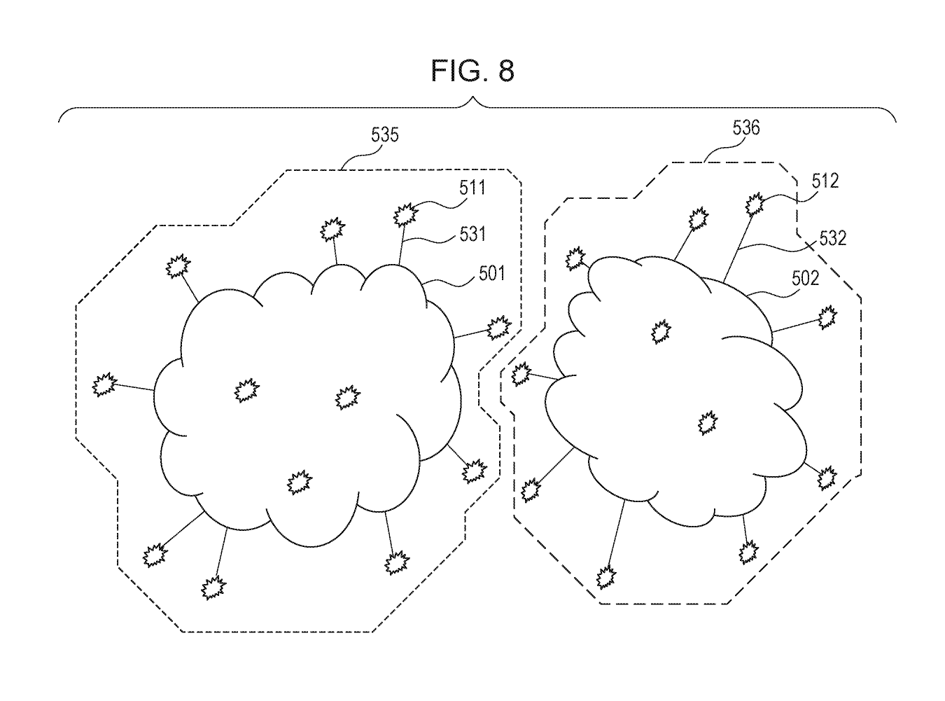

FIG. 8 illustrates one classification form in which the calcified tissue (the first tissue) and the tumor tissue (the second tissue) extracted by the extraction section 1228 are classified. In this case, the classification section 1230 classifies the calcified tissues based on the distance from the surface of the tumor tissue.

The classification section 1230 searches for the calcified tissue 511 using a distance 531 from the surface of the tumor tissue 501. Specifically, the classification section 1230 determines whether the distance 531 between the calcified tissue 511 and the surface of the tumor tissue 501 on the normal to the surface of the tumor tissue 501 is within a predetermined distance. For calcified tissues other than the calcified tissue 511, the classification section 1230 determines whether the distance between each of the calcified tissues and the surface of the tumor tissue 501 on the normal to the surface of the tumor tissue 501 is within a predetermined distance. When the classification section 1230 determines that the distance 531 between the surface of the tumor tissue 501 and the calcified tissue is within a predetermined distance, the classification section 1230 recognizes that the tumor tissue 501 and the calcified tissue are of the same group and sets the group as a group 535. In other words, the classification section 1230 can classify the group of the calcified tissue 511 (the first tissue) as the group 535 (a first group) with reference to the surface of the tumor tissue 501 (the second tissue).

Likewise, the classification section 1230 searches for the calcified tissue 512 using a distance 532 from the surface of the tumor tissue 502. Specifically, the classification section 1230 determines whether the distance 532 between the calcified tissue 512 and the surface of the tumor tissue 502 on the normal to the surface of the tumor tissue 502 is within a predetermined distance. For calcified tissues other than the calcified tissue 512, the classification section 1230 determines whether the distance between each of the calcified tissues and the surface of the tumor tissue 502 on the normal to the surface of the tumor tissue 502 is within a predetermined distance. When the classification section 1230 determines that the distance 532 between the surface of the tumor tissue 502 and the calcified tissue is within a predetermined distance, the classification section 1230 recognizes that the tumor tissue 502 and the calcified tissue are of the same group and sets the group as a group 536. In other words, the classification section 1230 can classify the group of the calcified tissue 512 (the first tissue) as the group 536 (a second group) with reference to the surface of the tumor tissue 502 (the second tissue).

The classification section 1230 outputs the information indicating the group 535 (the first group) of the calcified tissue 511 in the tumor tissue 501 and the information indicating the group 536 (the second group) of the calcified tissue 512 in the tumor tissue 502 to the display 124. The display 124 distinguishably displays the group 535 of the calcified tissue 511 in the tumor tissue 501 and the group 536 of the calcified tissue 512 in the tumor tissue 502. This allows the operator to ascertain the size of the specific tissue and the relationship between the specific tissue and the other tissue.

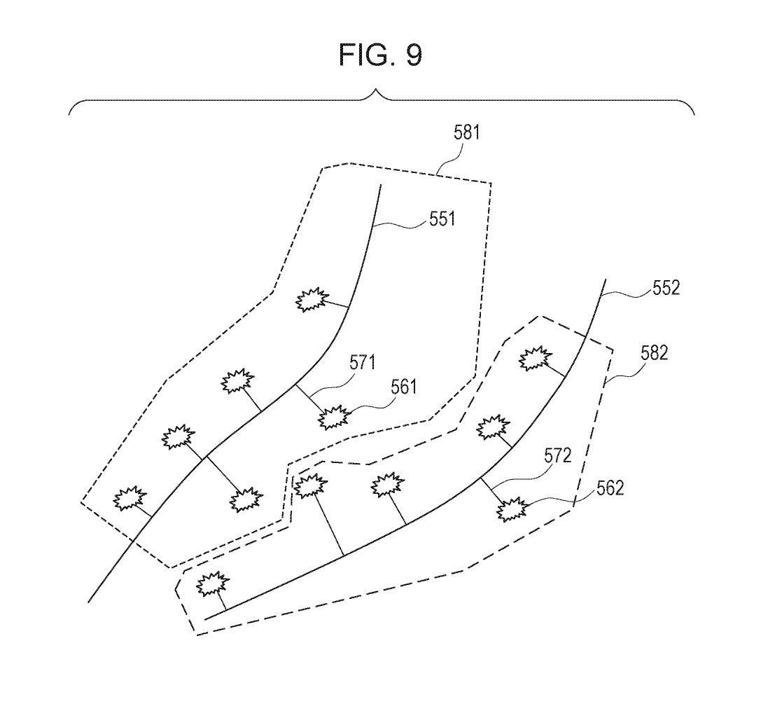

FIG. 9 illustrates one classification form in which the calcified tissue (the first tissue) extracted by the extraction section 1228 and other biological tissue are classified. This illustrates a case in which the groups of calcified tissues are determined based on a biological tissue with a luminal structure, such as a mammary gland or blood vessels.

Examples of the biological tissue with a luminal structure include continuously linked mammary glands and blood vessels. The extraction section 1228 analyzes the CT values of a three-dimensional image in three dimensions to extract a continuously linked tissue with a CT value lower than the CT values of the surroundings.

The classification section 1230 searches for calcified tissue using a distance from a biological tissue with a luminal structure. Specifically, the classification section 1230 determines whether a distance 571 between a calcified tissue 561 and the surface of a biological tissue 551 with a luminal structure is within a predetermined distance. For calcified tissues other than the calcified tissue 561, the classification section 1230 determines whether the distance between each of the calcified tissues and the surface of the biological tissue 551 with a luminal structure is within a predetermined distance. When the classification section 1230 determines that the distance 571 between the biological tissue 551 with a luminal structure and the calcified tissue is within a predetermined distance, the classification section 1230 recognizes that the biological tissue 551 and the calcified tissue are of the same group and sets the group as a group 581 (a first group).

Likewise, the classification section 1230 determines whether a distance 572 between a calcified tissue 562 and the surface of a biological tissue 552 with a luminal structure is within a predetermined distance. For calcified tissues other than the calcified tissue 562, the classification section 1230 determines whether the distance between each of the calcified tissues and the surface of the biological tissue 552 with a luminal structure is within a predetermined distance. When the classification section 1230 determines that the distance 572 between the biological tissue 552 with a luminal structure and the calcified tissue is within a predetermined distance, the classification section 1230 recognizes that the biological tissue 552 and the calcified tissue are of the same group and sets the group as a group 582 (a second group).

The classification section 1230 outputs the information indicating the group 581 (the first group) of the calcified tissue 561 in the tumor tissue 551 and the information indicating the group 582 (the second group) of the calcified tissue 562 in the biological tissue 552 with a luminal structure to the display 124. The display 124 distinguishably displays the group 581 of the calcified tissue 561 in the biological tissue 551 with a luminal structure and the group 582 of the calcified tissue 562 in the biological tissue 552 with a luminal structure. This allows the operator to ascertain the size of the specific tissue and the relationship between the specific tissue and the other tissue (the biological tissue with a luminal structure).

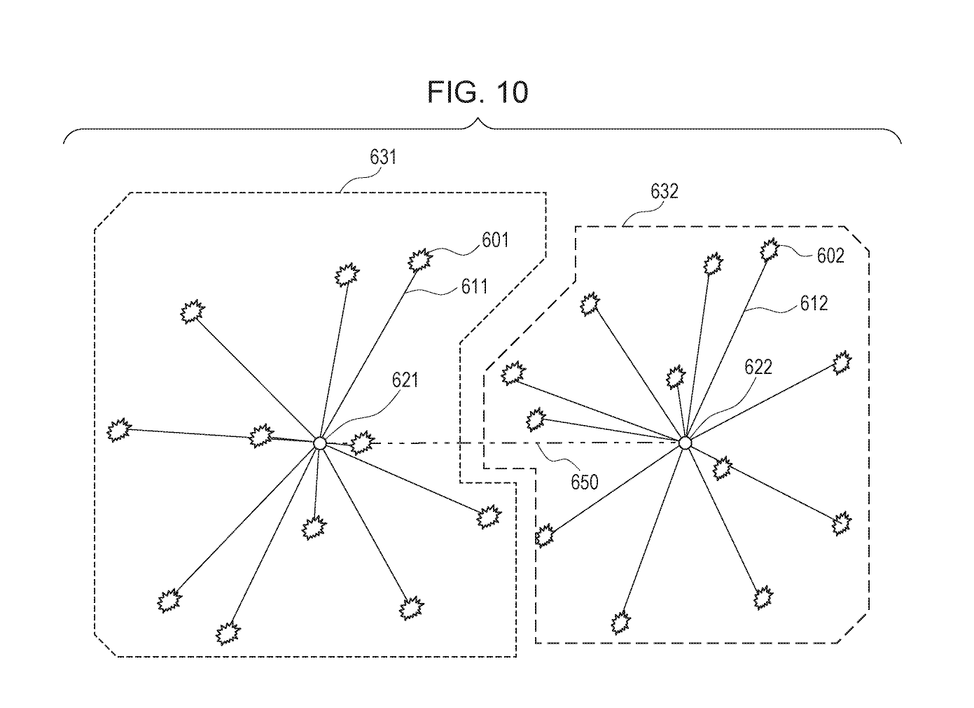

FIG. 10 illustrates one classification form in which calcified tissues (first tissues) extracted by the extraction section 1228 are classified.

The classification section 1230 classifies the calcified tissues into a plurality of groups using the distance between the groups of the calcified tissues (the first tissues), The classification section 1230 may classify the calcified tissues into a plurality of groups using a method of cluster analysis while changing the center of gravity of each group of the calcified tissues. Here, the classification section 1230 sets a central point 621 from the distribution information on the calcified tissues including a calcified tissue 601 at the left in FIG. 10 and a central point 622 from the distribution information on the calcified tissues including a calcified tissue 602 at the right in FIG. 10. To determine the number of clusters in the cluster analysis, a hierarchical cluster analysis method can be used. The hierarchical cluster analysis is an analysis of classifying groups in order from a combination of a plurality of calcified tissues. When an inter-group distance 650 between the central point 621 and the central point 622 which are set from the distribution information on the plurality of calcified tissues is longer than a preset threshold, the classification section 1230 may regard the calcified tissues are of different groups and may determine the number of groups. While examples of a method for obtaining the inter-group distance 650 include Ward's method, a group average method, a minimum distance method, and so on, any of the methods may be used.

The classification section 1230 outputs information indicating a calcified tissue group 631 including the calcified tissue 601 and information indicating a calcified tissue group 632 including the calcified tissue 602 to the display 124. The display 124 distinguishably displays the calcified tissue group 631 including the calcified tissue 601 and the calcified tissue group 632 including the calcified tissue 602. This allows the operator to ascertain the sizes of the calcified tissues.

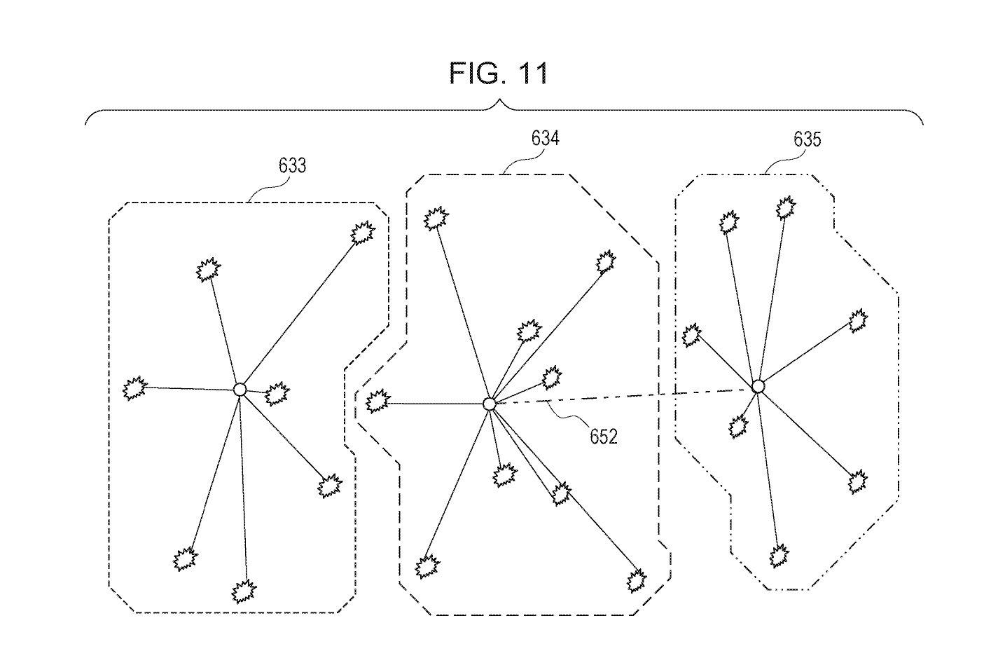

As illustrated in FIG. 11, the operator may change the threshold of the inter-group distance as appropriate while checking the classification after the cluster analysis. Although the distributions of the calcified tissues in FIG. 10 and FIG. 11 are the same, the classified states differ because the inter-group distance 650 and the inter-group distance 652 are appropriately changed. In the classified state illustrated in FIG. 11, the number of groups increases (groups 633, 634, and 635) as compared with the classified state illustrated in FIG. 10, but the inter-group distance may be changed so that the number of groups decreases. Alternatively, the classification section 1230 may classify the calcified tissues into a plurality of groups by adjusting the number of calcified tissues.

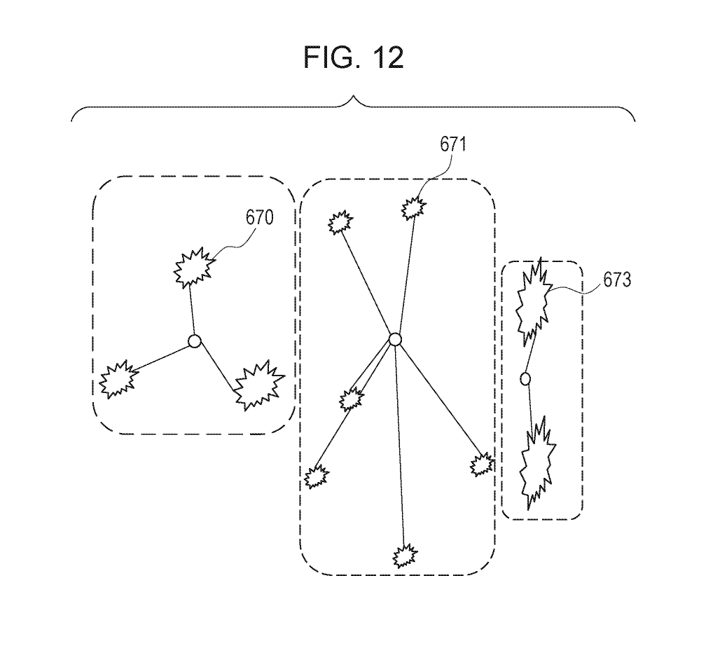

Alternatively, as illustrated in FIG. 12, the calcified tissues may be weighted. based on the size or the CT value of each calcified tissue. When the calcified tissue is large like a calcified tissue 670 and a calcified tissue 673, the classification section 1230 decreases the number of calcified tissues and classifies the calcified tissue groups. When the calcified tissue is small like a calcified tissue 671, the classification section 1230 increases the number of calcified tissues and classifies the calcified tissue groups. Since the calcified tissue groups are classified according to the size of the calcified tissues, the operator can easily distinguish the calcified tissue groups.

When the CT values of the calcified tissues are relatively high, the classification section 1230 may decrease the number of calcified tissues and classify the calcified tissue groups. When the CT values of the calcified tissues are low, the classification section 1230 increases the number of calcified tissues and classifies the calcified tissue groups. Since the calcified tissue groups are classified according to the CT values of the calcified tissues, the operator can easily distinguish the calcified tissue groups.

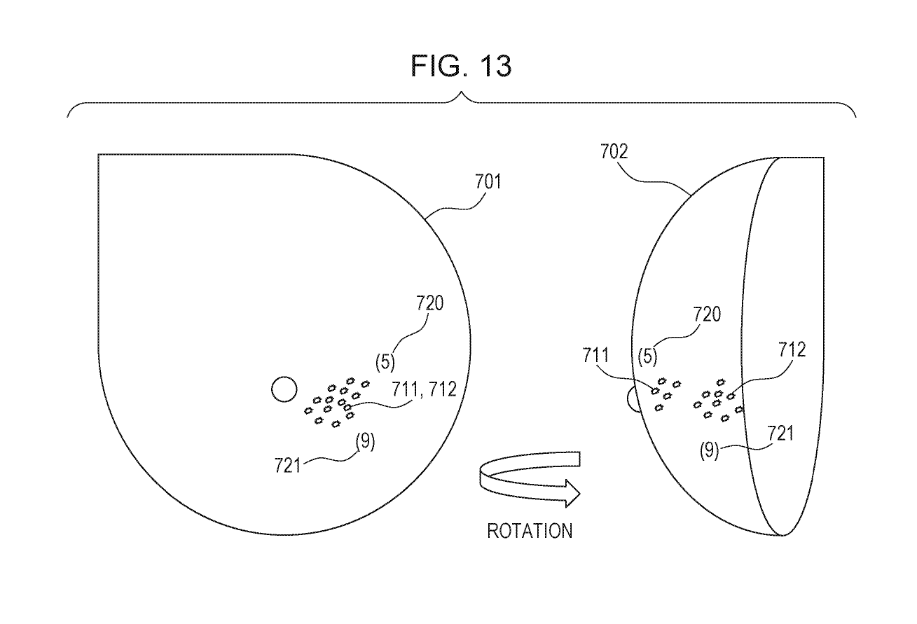

FIGS. 13 to 15 are diagrams illustrating display forms of the display 124. The display 124 displays a three-dimensional image based on the volume data reconstructed by the image processing unit 122 together with information indicating the groups of calcified tissues (the first tissues).

The display 124 may display a three-dimensional image of one breast or three-dimensional images of the right and left breasts of the subject captured at the same time side by side for comparison. The display 124 may display past and present three-dimensional images of the same regions side by side for comparison or may display the three-dimensional image together with a spot three-dimensional image of the same region for comparison. The display 124 may display the three-dimensional image and a three-dimensional image of another subject having a similar lesion for comparison.

FIG. 13 illustrates a three-dimensional image 701 and a three-dimensional image 702 obtained by rotating the three-dimensional image 701. The display 124 displays a calcified tissue 711 and a calcified tissue 712 extracted by the extraction section 1228 in the three-dimensional image 701. The display 124 displays information 720 (group information) indicating the group of the calcified tissue 711 and information 721 (group information) indicating the group of the calcified tissue 712 in the three-dimensional image 701. The information 720 indicating the group of the calcified tissue 711 is information indicating the number of calcified tissues including the calcified tissue 711. In the display form illustrated in FIG. 13, the number of calcified tissues including the calcified tissue 711 is “5”. The information 721 indicating the group of the calcified tissue 712 is information indicating the number of calcified tissues including the calcified tissue 712. In the display form illustrated in FIG. 13, the number of calcified tissues including the calcified tissue 712 is “9”.

The display 124 displays the information indicating the group of calcified tissues (the first tissues) in the three-dimensional image. Therefore, even when the three-dimensional image is rotated, the information (group information) indicating the calcified tissue group follows. This allows the operator to ascertain the size of the specific tissue.

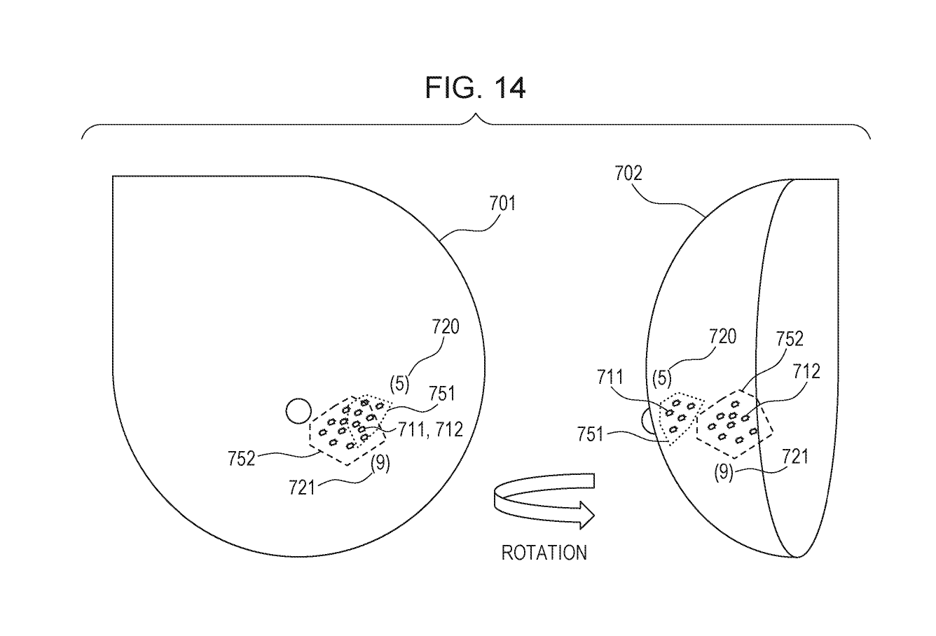

FIG. 14 illustrates the three-dimensional image 701 and the three-dimensional image 702 obtained by rotating the three-dimensional image 701. The display 124 displays the calcified tissue 711 and the calcified tissue 712 extracted by the extraction section 1228 in the three-dimensional image 701. Furthermore, the display 124 displays a region 751 (a three-dimensional region) indicating the group of the calcified. tissue 711 and a region 752 (a three-dimensional region) indicating the group of the calcified tissue 712 in the three-dimensional image 701. The display 124 displays the region 751 indicating the group of the calcified tissue 711 in blue, and the region 752 indicating the group of the calcified tissue 712 in red. The display 124 may display the region 751 indicating the group of the calcified tissue 711 with a thick frame (a solid line), and the region 752 indicating the group of the calcified tissue 712 with a thin frame (a broken line). The display 124 may have any display form in which the region 751 indicating the group of the calcified tissue 711 and the region 752 indicating the group of the calcified tissue 712 are displayed are distinguishably displayed.

The information 720 indicating the group of the calcified tissue 711 is information indicating the number of calcified tissues including the calcified tissue 711. The display 124 displays the information 720 (group information) indicating the group of the calcified tissue 711 and the region 751 (a three-dimensional region) indicating the group of the calcified tissue 711. The information 721 indicating the group of the calcified tissue 712 is information indicating the number of calcified tissues including the calcified tissue 712. The display 124 displays the information 721 (group information) indicating the group of the calcified tissue 712 and the region 752 (a three-dimensional region) indicating the group of the calcified tissue 712. This allows the operator to ascertain the size of a specific tissue in more detail from the information indicating the number of calcified tissues and the region.

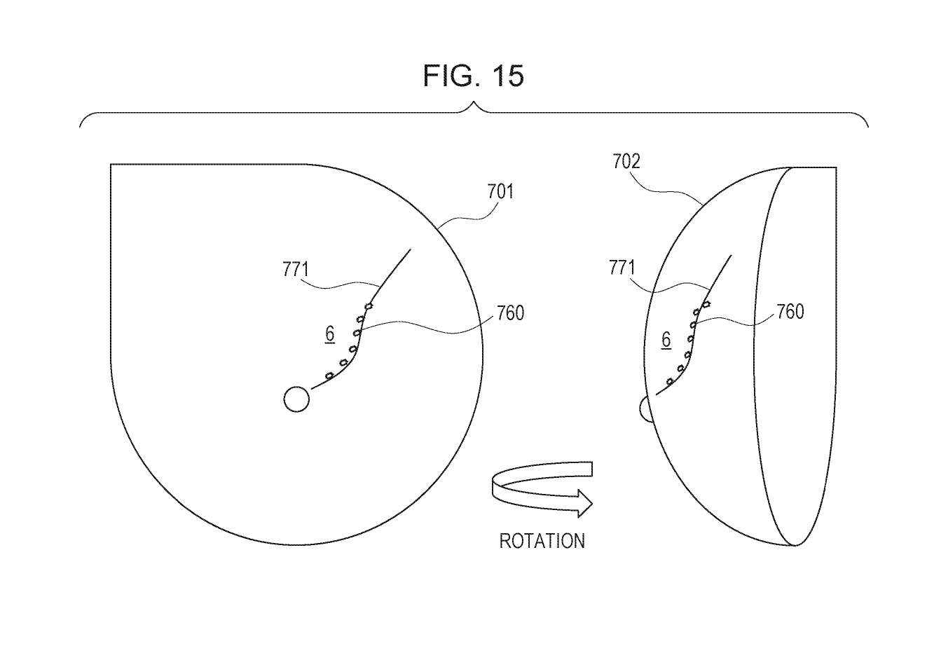

FIG. 15 illustrates the three-dimensional image 701 and the three-dimensional image 702 obtained by rotating the three-dimensional image 701. The three-dimensional image 701 includes a biological tissue 771 with a luminal structure, such as a mammary gland or blood vessels, and a calcified tissue group including a calcified tissue 760. In the vicinity of the biological tissue 771, information indicating the number of calcified tissues including the calcified tissue 760 is displayed.

Likewise, the three-dimensional image 702 obtained by rotating the three-dimensional image 701 includes the biological tissue 771 with a luminal structure, such as a mammary gland or blood vessels, and the calcified tissue group including the calcified tissue 760. In the vicinity of the biological tissue 771, information (group information) indicating the number of calcified tissues including the calcified tissue 760 is displayed. Specifically, the operator can ascertain the characteristics of the calcified tissues from the positional information on the biological tissue 771 with a luminal structure, such as a mammary gland or blood vessels, and the group of calcified tissues including the calcified tissue 760. For example, when the biological tissue 771 with a luminal structure and the group of calcified tissues including the calcified tissue 760 are close to each other, the calcified tissues are highly likely to be malignant. When the biological tissue 771 with the luminal structure and the group of calcified tissues including the calcified tissue 760 are far from each other, the calcified tissues are unlikely to be malignant Thus, the operator can ascertain the characteristics of the calcified tissues (the degree of malignancy) from the information indicating the number of calcified tissues and the biological tissue with the luminal structure.

The image processing apparatus (the image processing unit 122) according to an embodiment of the present disclosure includes the extraction section 1228 that extracts first tissues and second tissues from a three-dimensional image, the classification section 1230 that classifies the group of the first tissues with reference to the second tissues, and a display control unit (not illustrated) that causes the first tissue group classified by the classification section 1230 to be distinguishably displayed on the display 124.

A computer program for implementing the functions of the above embodiments can be supplied to a computer via a network or a storage medium (not illustrated) so as to be implemented. In other words, the computer program is a program for implementing the functions of the image processing apparatus with a computer. The storage medium stores the computer program.

### Other Embodiments

Embodiments of the present disclosure can also be realized by a computer of a system or apparatus that reads out and executes computer executable instructions recorded on a storage medium (e.g., non-transitory computer-readable storage medium) to perform the functions of one or more of the above-described embodiment(s) of the present disclosure, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While exemplary embodiments have been described, it is to be understood that the invention is not limited to the disclosed exemplary embodiments, The scope of the following claims is to be accorded the broadest interpretation so as to encompass all such modifications and equivalent structures and functions.

This application claims the benefit of Japanese Patent Application No. 2017-162616 filed Aug. 25, 2017, which is hereby incorporated by reference herein in its entirety.

What is claimed is:

1. A breast imaging apparatus comprising:
a gantry including a radiation generating unit configured to generate radiation and a radiation detecting unit configured to detect the radiation emitted from the radiation generating unit, wherein the radiation generating unit and the radiation detecting unit can be rotated in an opposed state;
an extraction unit configured to extract first tissues and a second tissue from a three-dimensional image based on a projection image output from the radiation detecting unit;
a classification unit configured to classify a group of the first tissues with reference to the second tissue; and
a display configured to distinguishably display the group of the first tissues classified by the classification unit.

2. The breast imaging apparatus according to claim 1, further comprising:
a specification unit configured to specify a position corresponding to the second tissue in the three-dimensional image,
wherein the classification unit classifies the group of the first tissues with reference to the specified position.

3. The breast imaging apparatus according to claim 2, wherein the classification unit searches for the first tissues based on a distance from the specified position.

4. The breast imaging apparatus according to claim 2,
wherein the classification unit determines whether a distance between the specified position and each first tissue is within a predetermined distance, and
wherein, in a case where it is determined that the distance is within the predetermined distance, the classification unit classifies the first tissues as a same group.

5. The breast imaging apparatus according to claim 1, wherein the classification unit searches for the first tissues based on a distance from a surface of the second tissue.

6. The classification unit according to claim 1, wherein the classification unit classifies the group of the first tissues based on a preset distance or number.

7. The breast imaging apparatus according to claim 1, wherein the display displays the three-dimensional image together with information indicating the group of the first tissues.

8. The breast imaging apparatus according to claim 1, wherein the display distinguishably displays a first group of the first tissues and a second group of the first tissues extracted with reference to a plurality of second tissues.

9. The breast imaging apparatus according to claim 1, wherein a method for extracting the first tissues and a method for extracting the second tissue with the extraction unit differ from each other.

10. The breast imaging apparatus according to claim 1, wherein the first tissues are calcified tissues, and the second tissue is a tumor tissue or a tissue with a luminal structure.

11. An image processing apparatus comprising:
an extraction unit configured to extract first tissues and a second tissue from a three-dimensional image;
a classification unit configured to classify a group of the first tissues with reference to the second tissue; and
a display control unit configured to distinguishably display the group of the first tissues classified by the classification unit on a display.

12. A method of image processing, the method comprising:
extracting first tissues and a second tissue from a three-dimensional image;
classifying a group of the first tissues with reference to the second tissue; and
distinguishably displaying the group of the first tissues classified by the classification unit on a display.
